(19) United States
(12) Patent Application Publication
(10) Pub. No.: US 2024/0139051 A1
(43) Pub. Date: May 2, 2024

(54) BRAKING DEVICE AND EXAMINATION BED

(71) Applicant: SHANGHAI UNITED IMAGING HEALTHCARE CO., LTD., Shanghai (CN)

(72) Inventors: HONG-AN ZHANG, Shanghai (CN); JIAN-NING ZHANG, Shanghai (CN)

(21) Appl. No.: 18/384,851

(22) Filed: Oct. 28, 2023

(30) Foreign Application Priority Data
Oct. 28, 2022 (CN) 202222861500.7

(51) Int. Cl. A61G 7/05; A61G 7/002

(52) U.S. Cl. CPC A61G 7/0528; A61G 7/002

(57) ABSTRACT

A braking device includes a wheel frame plate assembly, a first connecting member connected to the wheel frame plate assembly, and an electromagnet assembly. The wheel frame plate includes a wheel frame plate and a wheel, one side of the wheel frame plate along a first direction is connected to the wheel, and the wheel is rotatable and adapted to be disposed above a braking plate. The electromagnet assembly includes an electromagnet connected to the wheel frame plate and being rotatable around a radial direction of the first connecting member, and the electromagnet and the wheel is located on the same side of the wheel frame plate along the first direction. The electromagnet is adapted to be attracted to or separated from the braking plate by moving along an axial direction of the first connecting member.

### CROSS-REFERENCE TO RELATED APPLICATIONS

This application claims priority of Chinese Patent Application No. 2022228615007, filed on Oct. 28, 2022, the content of which is hereby incorporated by reference in its entirety.

### TECHNICAL FIELD

The present disclosure relates to medical devices, in particular to a braking device and an examination bed.

### BACKGROUND

In some medical imaging and treatment equipment, an examination bed can move along a track under an operation of a medical staff to locate a patient's treatment site into a medical imaging device and/or a treatment device to capture an image or perform a treatment. In order to ensure the safety of the patient on a bed plate during the movement of the examination bed and to stop the bed plate in time after the bed plate is in place, a braking device is required to be provided in the examination bed to control the movement of the examination bed.

In related art, the braking device of an examination bed generally adopts an electromagnetic brake. The electromagnetic brake is located under the bed plate and can be attached to and separated from a braking plate provided on a bed frame. When the electromagnetic brake is demagnetized, the electromagnetic brake can be driven to move along with the bed plate. When the electromagnetic brake is magnetized, the electromagnetic brake and the braking plate are magnetically attracted. Due to the magnetic attraction between the electromagnetic brake and the braking plate, the electromagnetic brake cannot be driven to move along with the bed plate, so that the bed plate is braked and stop moving.

However, when the braking plate is not parallel to electromagnetic brake, there is only a small contact area between the electromagnetic brake and the braking plate, resulting in insufficient magnetic attraction. The bed plate can easily overcome the magnetic attraction and continue to move, which means that the braking of the bed plate fails, resulting in a decrease in the safety performance of the examination bed, which brings potential risk to the safety of patients.

### SUMMARY

An object of the present disclosure is to provide a braking device and an examination bed.

The present disclosure provides a braking device, which includes a wheel frame plate assembly, a first connecting member connected to the wheel frame plate assembly, and an electromagnet assembly. The wheel frame plate assembly includes a wheel frame plate and a wheel, one side of the wheel frame plate along a first direction is connected to the wheel, and the wheel is rotatable and adapted to be disposed above a braking plate. The electromagnet assembly includes an electromagnet, the electromagnet is connected to the wheel frame plate and is rotatable around a radial direction of the first connecting member, and the electromagnet and the wheel being located on the same side of the wheel frame plate along the first direction; wherein the electromagnet is adapted to be attracted to or separated from the braking plate by moving along an axial direction of the first connecting member, so as to brake or release braking force on the braking plate.

In one of the embodiments, the braking device further includes a rotating shaft, the rotating shaft extends through the first connecting member in a direction perpendicular to the first connecting member, and both ends of the rotating shaft are respectively connected to the wheel frame plate.

In one of the embodiments, the braking device further includes a first elastic assembly, the first elastic assembly includes a second connecting member and a first elastic member, the second connecting member movably extends through the wheel frame plate, a bottom portion of the second connecting member is connected to the electromagnet, and the first elastic member deforms or resets due to the movement of the electromagnet in a direction closer to or away from the braking plate.

In one of the embodiments, a top portion of the second connecting member is provided with a first protruding portion, and the first protruding portion protrudes outward around a circumferential direction of the second connecting member. One end of the first elastic member abuts against the first protruding portion, and the other end of the first elastic member is connected to the wheel frame plate.

In one of the embodiments, the braking device has a power-off state and a power-on state. When the braking device is in the power-off state, the electromagnet is magnetized and is attracted to the braking plate, and the first elastic member is compressed and stores potential energy; and when the braking device is in the power-on state, the electromagnet demagnetizes, and separate from the braking plate, and the first elastic member stretches and releases potential energy, thereby driving the electromagnet to move away from the braking plate.

In one of the embodiments, the braking device further includes a housing and a second elastic assembly, the second elastic assembly includes a third connecting member and a second elastic member, the first connecting member extends through the wheel frame plate, the housing covers a portion of the first connecting member located above the wheel frame plate. The third connecting member extends through the housing, and is in contact with a top portion of the first connecting member. The second elastic member is sleeved on the third connecting member, and the second elastic member deforms or resets due to the movement of the electromagnet in a direction closer to or away from the braking plate.

In one of the embodiments, a top portion of the third connecting member has a second protruding portion, the second protruding portion protrudes outward around a circumferential direction of the third connecting member, one end of the second elastic member abuts against the second protruding portion, and the other end of the second elastic member is connected to the first connecting member.

In one of the embodiments, the braking device has a power-off state and a power-on state. When the braking device is in the power-off state, the electromagnet is magnetized, and the second elastic member is compressed and stores potential energy; and when the braking device is in the power-on state, the electromagnet is demagnetized, and separate from the braking plate, the second elastic member stretches and releases potential energy, thereby driving the electromagnet to move away from the braking plate.

In one of the embodiments, the braking device further includes a mounting base. The mounting base is arranged between the wheel frame plate and the electromagnet and is configured to be mounted to the electromagnet, and the second connecting member extends through the mounting base and is in contact with the electromagnet. The wheel frame plate assembly further includes a wheel frame, a connecting shaft, a snap spring, and a washer. The wheel frame extends along the first direction, one end of the wheel frame is connected to the wheel frame plate, another end of the wheel frame is provided with a recessed groove extending along the first direction, the connecting shaft extends through the recessed groove along a second direction, the wheel is sleeved on the connecting shaft, the connecting shaft extends through the groove along a direction perpendicular to the second direction. The wheel and the washer are sequentially sleeved on the connecting shaft, and the snap ring is arranged on a side wall of the wheel frame and is engaged with the connecting shaft.

The present disclosure further provides a braking device, which includes a wheel frame plate assembly, a first elastic assembly and an electromagnet assembly. The frame plate assembly includes a wheel frame plate and a wheel, one side of the wheel frame plate along a first direction is connected to the wheel, and the wheel is rotatable and adapted to be disposed above a braking plate. The electromagnet assembly is connected to the wheel frame plate through the first elastic assembly, and the electromagnet assembly and the wheel are located on the same side of the wheel frame plate. The first elastic assembly is capable of deforming or resetting based on movement of the electromagnet assembly in a direction close to or away from the braking plate, so as to enable the braking device to complete braking or release braking.

In one of the embodiments, the braking device further includes a first connecting member and a rotating shaft. The rotating shaft extends through the first connecting member in a direction perpendicular to the first connecting member, and both ends of the rotating shaft are connected to the wheel frame plate respectively to enable the first connecting member and the wheel frame plate to be rotatable with each other.

In order to achieve the above purpose, the present disclosure further provides an examination bed, including a bed frame, a bed plate assembly, and the braking device as described above. The braking plate is fixed inside the bed frame, the braking device is located between the bed plate assembly and the braking plate, and the braking device is fixed to the bed plate assembly through a fastener (e.g., a bolt, a screw, etc.).

The braking device provided by embodiments of the present disclosure can achieve following benefits:
(1) When the bed plate assembly and the braking plate are not parallel, the first connecting member is connected to the bed plate assembly through the housing and the connecting plate, and the first connecting member is rotatably connected to the wheel frame plate through the rotating shaft, so the first connecting member can drive the bed plate assembly to rotate around the rotating shaft by a certain angle, and/or the wheel frame plate is rotated around the rotating shaft by a certain angle, and the bed plate assembly can be adjusted to be parallel to the braking plate from being non-parallel to the braking plate. When the electromagnet and the braking plate are magnetically attracted, the contact area between the electromagnet and the braking plate can be increased, further increasing a braking force provided by the braking device on the braking plate.
(2) When the electromagnet is magnetized, the electromagnet is pulled toward the braking plate due to the attraction, thereby the first elastic member deforms, so that the electromagnet moves downward and attracts the braking plate to complete the braking. When the electromagnet is demagnetized, the first elastic member is reset, and the electromagnet is driven to be lifted and reset, so that the electromagnet and is separated from the braking plate, thereby releasing the braking. Since the position of the electromagnet after being lifted and reset is higher than the position of the roller, there will be no motion friction due to contact between the electromagnet and the braking plate.

### BRIEF DESCRIPTION OF THE DRAWINGS

FIG. 1 is a perspective view of a braking device according to an embodiment of the present disclosure.

FIG. 2 is a partial schematic view of the braking device according to an embodiment of the present disclosure.

FIG. 3 is a partial front view of the braking device of FIG. 1 without showing a housing and a connecting plate.

FIG. 4 is a partial front view of the braking device of FIG. 3 without showing a third connecting member.

FIG. 5 is a cross-sectional view taken along line A-A of FIG. 4.

FIG. 6 is a partial perspective view of the braking device according to an embodiment of the present disclosure without showing the housing and the connecting plate.

FIG. 7 is a partial front view of the braking device of FIG. 3 without showing an electromagnet and a mounting base.

FIG. 8 is a cross-sectional view taken along line B-B of FIG. 7.

FIG. 9 is a perspective view of a wheel assembly according to an embodiment of the present disclosure.

FIG. 10 is a schematic view of an examination bed according to an embodiment of the present disclosure.

### DESCRIPTION OF REFERENCE SIGNS

1—electromagnet; 2—wheel; 3—wheel frame plate; 4—housing; 5—mounting base;

11—first connecting member; 12—rotating shaft; 13—second connecting member; 14—first elastic member; 15—third connecting member; 16—second elastic member;

21—wheel frame; 22—connecting shaft; 23—snap spring; 24—washer;

31—first through hole; 32—first groove; 33—first accommodation hole; 34—second accommodation hole; 111—third accommodation hole;

131—first protruding portion; 141—first segment; 142—second segment; 151—second protruding portion; 161—third segment; 162—fourth segment;

Z—first direction; Y—second direction; X—third direction;

6—bed frame; 7—bed plate assembly; 8—braking device 8; 9—braking plate; 10—connecting plate 71—moving member; 72—bed plate.

### DETAILED DESCRIPTION OF THE EMBODIMENTS

In order to make the objects, advantages and features of the present disclosure clearer, the present disclosure will be further described in detail below in conjunction with the accompanying drawings and specific embodiments. It should be noted that the drawings are all in a very simplified form and not drawn to scale, and are only used to facilitate and clearly assist the purpose of illustrating the embodiments of the present disclosure. In addition, the structures shown in the drawings are often a part of the actual structure. In particular, different drawings may focus on different matters, and different scales may be used.

As used in this specification, the singular forms “a”, “an” and “the” include plural objects, the term “or” is usually used in the sense of including “and/or”, and the term “several” is usually used in the meaning including “at least one”, and the term “at least two” is usually used in the meaning including “two or more”. In addition, the terms “first”, “second”, and “third” are used for descriptive purposes only, and should not be understood as indicating or implying relative importance or implicitly specifying the number of indicated technical features. Thus, the features defined as “first”, “second”, and “third” may expressly or implicitly include one or at least two of these features. “One end” vs. “the other end” and “proximal end” vs. “distal end” generally refer to corresponding two portions, which include not only the end points. The terms “mounted”, “coupled”, and “connected” should be understood in a broad sense, for example, they may be fixedly connected or detachably connected, or integrally formed, or may be directly connected or indirectly connected through an intermediate element, and they can refer to the internal communication of two elements or the interaction relationship between two elements. In addition, as used in this specification, an element is arranged on another element, usually only means that there is a connection, coupling, cooperation or transmission relationship between the two elements, and the two elements may be directly or indirectly connected, coupled, engaged or driven through an intermediate element, and should not be understood as indicating or implying the spatial positional relationship between two elements. That is, an element may be in any orientation such as inside, outside, above, below or on a side of another element, unless otherwise expressly stated in the content. The terms “upper”, “lower”, “top”, and “bottom” usually refer to the relative positional relationship arranged in the direction of gravity; the term “vertical direction” usually refers to the direction of gravity, which is generally perpendicular to the ground, “horizontal direction” generally refers to a direction parallel to the ground. Those skilled in the art can understand the specific meanings of the above terms in this specification according to specific situations.

An object of embodiments of the present disclosure is to provide a braking device 8 and an examination bed to address a problem of insufficient braking force caused by a relatively small contact area between a magnet and a braking plate 9, resulting in a magnet being unable to completely attracted to the braking plate 9.

The present disclosure will be described below with reference to the accompanying drawings.

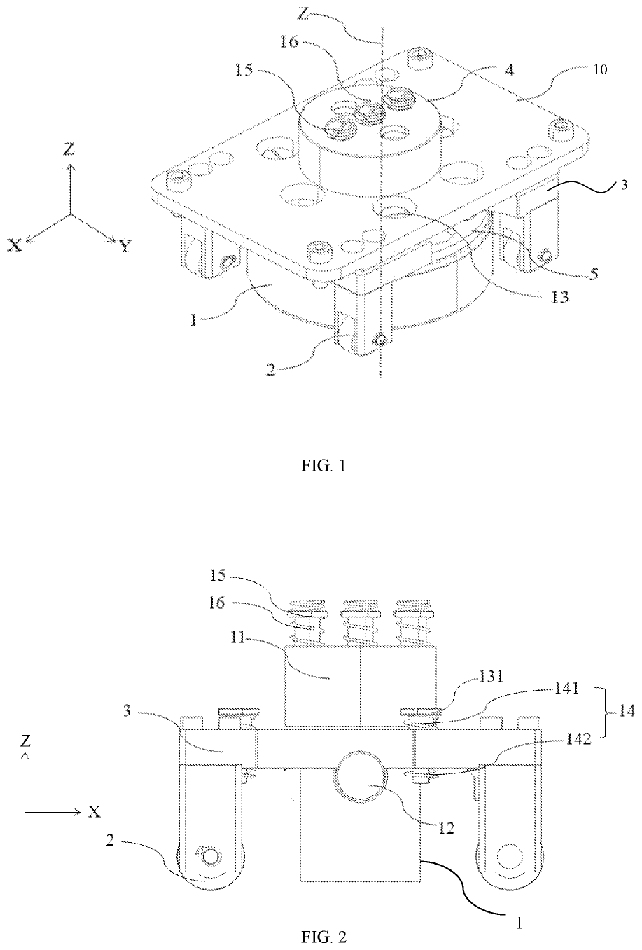

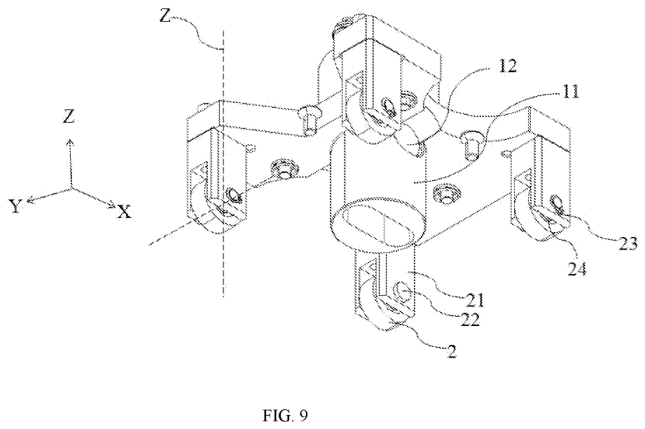

Referring to FIG. 1 to FIG. 2, an embodiment of the present disclosure provides a braking device 8. Referring to FIG. 9, the braking device 8 is configured to be mounted on an examination bed. The examination bed includes a bed frame 6, a bed plate assembly 7, a braking plate 9. The braking device 8 is provided between the bed plate assembly 7 and the braking plate 9. The braking plate 9 is provided on the bed frame 6. The bed plate assembly 7 is capable of moving back and forth on the bed frame 6, so as to drive the braking device 8 to move back and forth on the bed frame 6.

Referring to FIG. 9, the bed plate assembly 7 includes a moving member 71 and a bed plate 72. The moving member 71 drives the bed plate 72 to move horizontally on the bed frame 6. In this case, the braking device 8 may be mounted on the moving member 71 to brake the moving member 71, thereby braking the bed plate 72. In an embodiment, the bed plate assembly 7 may alternatively only include the bed plate 72, and the bed plate 72 is driven to move horizontally on the bed frame 6. In this case, the braking device 8 may be mounted on the bed plate 72, so that the braking device 8 brakes the bed plate 72.

The braking device 8 includes a connecting plate 10, a first connecting member 11, a wheel frame plate assembly, and an electromagnet assembly. The connecting plate 10 is configured to connect to the bed plate assembly 7, so as to drive the bed plate assembly 7 to move on the bed frame 6 along with braking device 8. The first connecting member 11 is connected to the wheel frame plate assembly. The electromagnet 1 is connected to a side of the wheel frame plate assembly away from the connecting plate 10 an is rotatable around a radial direction of the first connecting member 11.

Specifically, the wheel frame plate assembly includes a wheel frame plate 3 and a wheel 2. A first side of the wheel frame plate 3 along a first direction Z (e.g., a lower side in FIG. 2) is connected to the wheel 2, a second side of the wheel frame plate 3 (e.g., an upper side in FIG. 2) is connected to the first connecting member 11 through a rotating shaft 12, so that the wheel frame plate assembly and the first connecting member 11 can rotate relative to each other. The wheel 2 is rotatable and adapted to be disposed above the braking plate 9. The first direction Z is perpendicular to a plane where the wheel frame plate 3 is located. The electromagnet assembly includes an electromagnet 1. The wheel frame plate assembly further includes a connecting column 18 provided at the center of a lower side of the wheel frame plate 3. The electromagnet assembly is sleeved on the outer side of the connecting column 18 and connected to the wheel frame plate 3, so that the electromagnet assembly is connected to the wheel frame plate 3 and can rotate around an axis perpendicular to the first direction Z. In the embodiment, the electromagnet assembly further includes a mounting base 5. The mounting base 5 is provided on the lower side of the wheel frame plate 3 and is sleeved the outer side the connecting column 18. The electromagnet 1 is fixed to the mounting base 5. The braking device 8 further includes a first elastic assembly. The first elastic member is fixed to the mounting base 5, so that the electromagnet 1 can be connected to the wheel frame plate 3.

The wheel frame plate 3 can drive the electromagnet 1 to rotate within a preset angle range with the rotating shaft 12 as a rotating axis. That is, the electromagnet 1 can swing around the axis perpendicular to the first direction Z within the preset angle range. The electromagnet 1 can rotate around the axis perpendicular to the first direction Z within a preset angle range, such as −10 degrees to 10 degrees. The preset angle range may be in a range from −10 degrees to 10 degrees, which is not limited herein.

The electromagnet 1 and the wheel 2 are located on the same side of the wheel frame plate 3 along the first direction Z. The electromagnet 1 is adapted to be attracted to or separated from the braking plate 9 by moving along an axial direction of the first connecting member 11, so as to brake or release the braking force applied on the braking plate 9. In an embodiment, when the electromagnet 1 is energized (e.g., powered on), the electromagnet 1 moves away from the braking plate 9 to release the braking, and when the electromagnet 1 is de-energized (e.g., powered off), the electromagnet 1 moves towards the braking plate 9 to achieve braking. It should be noted that the material of the braking plate 9 is adapted to the electromagnet 1, so that the electromagnet 1 can attract the braking plate 9.

In this embodiment, the electromagnet 1 is an electro permanent magnet, which demagnetizes when energized, and recovers its original magnetization when de-energized. The electromagnet 1 can swing around the axis perpendicular to the first direction Z, that is, the electromagnet 1 is rotatable around a rotation axis of the first connecting member 11. The rotation axis of the first connecting member 11 is parallel to the plane where the first connecting member 11 locates. A mounting surface of the braking device 8 9 is an upper surface of the connecting plate 10 in FIG. 1, which is configured to be connected to the bed plate assembly 7. The first direction Z is a height adjustment direction of the examination bed, which is perpendicular to the plane where the connecting plate 10 is located. A second direction Y and a third direction X are both perpendicular to the first direction Z. The second direction Y is also perpendicular to a moving direction of the examination bed, and is parallel to a plane on which the examination bed moves. The third direction X is the moving direction of the examination bed. With such configuration, when the braking device 8 is mounted to the examination bed, the electromagnet 1 can move along the axial direction of the first connecting member 11, so as to achieve braking and releasing of the braking through the attraction and separation of the electromagnet 1 and the braking plate 9. The axial direction of the first connecting member 11 can be perpendicular to the rotation axis of the first connecting member 11. When the mounting surface of the braking device 8 and the braking plate 9 have different relative positions, for example, when the mounting surface of the braking device 8 is not parallel to the braking plate 9, that is, when the braking device 8 is tilted relative to the braking plate 9, the electromagnet 1 can rotate around the axis perpendicular to the first direction Z, e.g., around the rotation axis of the first connecting member 11, to adapt to different relative positions of the mounting surface and the braking plate 9, so that a surface of the electromagnet 1 facing the braking plate 9 gradually come into contact with the braking plate 9 during the rotation of the electromagnet 1, i.e., the braking device 8 gradually becomes parallel to the braking plate 9, thereby increasing the contact area between the electromagnet 1 and the braking plate 9, and increasing a braking force provided by the braking device 8 on the braking plate 9. In some embodiments, the electromagnet 1 can rotate around the axis parallel to the second direction Y, e.g., the rotation axis of the first connecting member 11 is parallel to the second direction Y. It should be noted here that the movement of the electromagnet 1 is not limited to rotating around the rotation axis of the first connecting member 11, as long as the movement of the electromagnet 1 can be made such that the electromagnet 1 is fully adherent to the braking plate 9, i.e. the mounting surface is parallel to the braking plate 9.

As an optional embodiment, the braking device 8 further includes a rotating shaft 12, and the rotating shaft 12 extends through the first connecting member 11 in a direction perpendicular to the first connecting member 11. The rotating shaft 12 is rotatable relative to the wheel frame plate 3, and both ends of the rotating shaft 12 are respectively connected to the wheel frame plate 3, and the wheel frame plate 3 and the electromagnet 1 can rotate around the rotating shaft 12. FIG. 2 is a partial schematic view of the braking device 8 of FIG. 1 with a housing 4, a mounting base 5, the electromagnet 1 and a connecting plate 10 removed. Referring to FIG. 8 and FIG. 9, the rotating shaft 12 extends through the first connecting member 11 and a portion of the wheel frame plate assembly in an axis perpendicular to the first direction Z, e.g., in the rotation axis of the first connecting member 11.

Specifically, the wheel frame plate assembly is provided with a first accommodation hole 31 in the first direction Z and a second accommodation hole 34 in the second direction Y. The first accommodation hole 33 is in communication with the second accommodation hole 34. An end of the first connecting member 11 is provided with a third accommodation hole 111 in the second direction Y. The first connecting member 11 is located in the first accommodation hole 31 to align the third accommodation hole 111 with the second accommodation hole 34, then the rotating shaft 12 extends through the third accommodating hole 111 and the second accommodating hole 32, so that the first connecting member 11 is connected to the wheel frame assembly. The rotating shaft 12 is movably placed in the second accommodating hole 32 and the third accommodating hole 111, so that both the wheel frame plate assembly and the first connecting member 11 can rotate around the rotating shaft. 12. In an embodiment, one part of the rotating shaft 12 in the radial direction thereof is accommodated in the wheel frame plate 3, the other part of the rotating shaft 12 is accommodated in the connecting column 18, and both ends of the other part of the rotating shaft 12 are blocked by mounting base in the axial direction of the rotating shaft 12. In this way, the rotating shaft 12 is limited between the wheel frame plate 3 and the connecting column 18 to prevent the rotating shaft 12 from coming out.

When the braking device 8 brakes the braking plate 9 and the mounting surface and the braking plate 9 are not parallel, the electromagnet 1 has a tendency to be attached to the braking plate 9 due to the attraction force between the electromagnet 1 and the braking plate 9. Since the wheel frame plate assembly connected to the electromagnet 1 through the mounting base 5 can rotate around the rotating shaft 12, the electromagnet 1 can swing around the rotating axis 12 close to the braking plate 9, so as to drive the wheel frame plate assembly and the first connecting member 12 to swing along with the electromagnet 1 until the electromagnet 1 is completely attached to the braking plate 9. In this way, the contact area between the electromagnet 1 and the braking plate 9 can be increased and the braking force on the braking plate 9 can be further increased. In some embodiments, the entire rotating shaft 12 can alternatively be arranged on the wheel frame plate 3, or the first connecting member 11 can also be rotated by other components with the same function. Those skilled in the art can configure the rotating shaft 12 according to the actual situation, which is not limited herein. In other embodiments, the rotating shaft 12 may be replaced by a ball joint structure, so that the electromagnet 1 can be rotated in more directions as required.

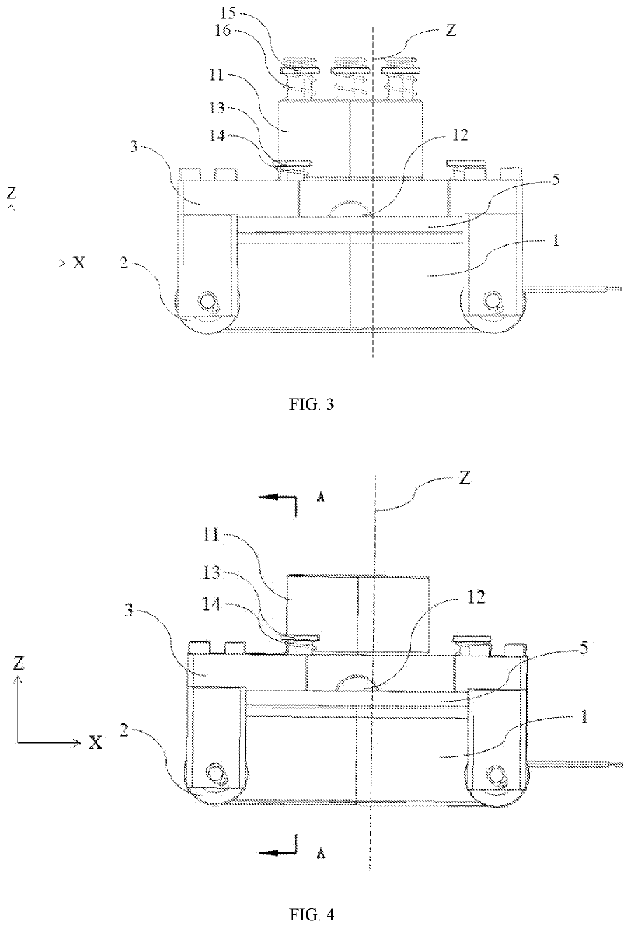

Referring to FIG. 3 to FIG. 5, in some embodiments, the braking device 8 further includes a first elastic assembly, and the first elastic assembly includes a second connecting member 13 and a first elastic member 14. The second connecting member 13 movably extends through the wheel frame plate 3, and a bottom portion of the second connecting member 13 is connected to the mounting base 5. In this way, the mounting base 5 is limited between the electromagnet 1 and the wheel frame plate 3, so that the mounting base 5 can move along with the electromagnet 1.

In an embodiment, the mounting base 5 is a flange providing a plurality of through holes and a plurality of fixing holes. The electromagnet 1 is sleeved on the bottom portion of the flange and is provided with a plurality of connecting holes aligned with the through holes. A plurality of fixing members extend through the plurality of through holes and are fixed in the connecting holes to fix the electromagnet and the mounting base 5. A plurality of second connecting members 13 are fixed in the plurality of fixing holes, so that the mounting base 5 is fixed to the second connecting members 13.

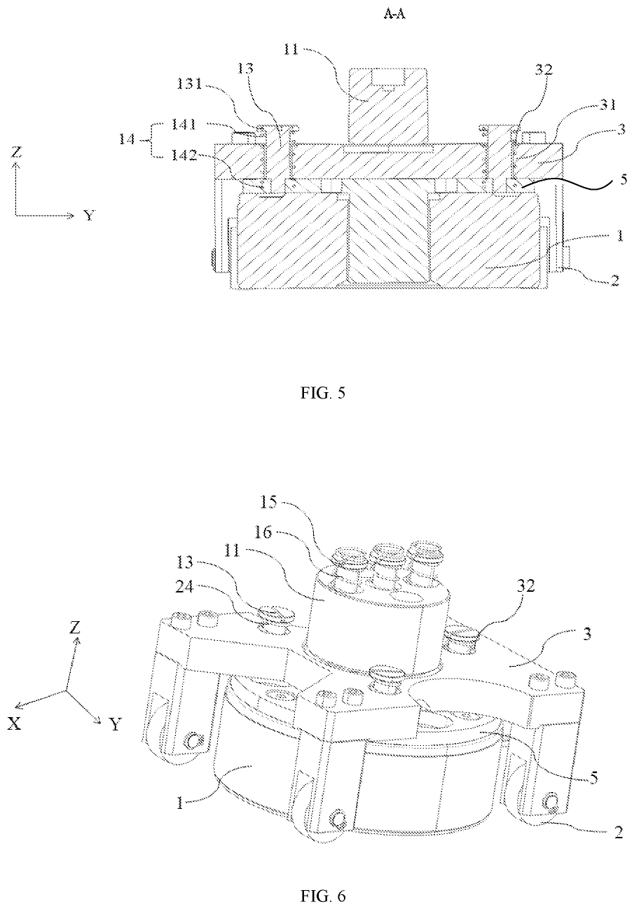

The first elastic member 14 is sleeved on the second connecting member 13, and the first elastic member 14 is configured to be deformed when the electromagnet 1 is powered off and magnetized to attract the braking plate 9, so as to allow the electromagnet 1 to be in contact with the braking plate 9, and provide elastic restoring force to enable the electromagnet 1 to move away from the braking plate 9 when the electromagnet is powered on and demagnetized, so as to separate the electromagnet 1 from the braking plate 9. FIG. 3 is a partial front view of the braking device 8 of FIG. 1 omitting the housing 4 and the connecting plate 6. Referring to FIG. 3 and FIG. 5, the first elastic member 14 is sleeved on the second connecting member 13, a portion of the first elastic member 14 is wound around an upper portion of the second connecting member 13, and another portion of the first elastic member 14 and the second connecting member 13 extend into a first groove 32 formed in the wheel frame plate 3 and extend through the wheel frame plate 3 to connected to the mounting base 5. Since the electromagnet 1 is fixed to the mounting base 5, the second connecting member is indirectly connected to the electromagnet 1. The first elastic member 14 utilizes its elasticity to cooperate with the mounting base 5 to lift or put down the mounting base 5, so that the electromagnet 1 is separated from or is in contact with the braking plate 9.

In another embodiment, the first elastic member 14 may be sleeved on a rod portion of the second connecting member 13. One end of the first elastic member 14 abuts against a head portion of the second connecting member 13, and the other end of the first elastic member 14 is located in the first groove 32 and abuts against a bottom wall of the first groove 32, so as to achieve the cooperation between the first elastic member 14 and the wheel frame plate 3. Since the electromagnet 1 is rotatable around the radial direction of the first connecting member 11, when the electromagnet 1 is in contact with the braking plate 9, the contact area between the electromagnet 1 and the braking plate 9 can be increased through the rotation of the electromagnet 1, thereby further increasing the braking force exerted on the braking plate 9. In an embodiment, the braking device 8 includes a plurality of the first elastic assemblies, and the plurality of the first elastic assemblies are evenly spaced around a circumference of the electromagnet assembly. With such configuration, the plurality of first elastic assemblies can apply force to the electromagnet 1 evenly, so that the electromagnet 1 can be pulled up or lowered smoothly. In the embodiment shown in FIG. 6, the number of the first elastic assemblies is three, in some embodiments, the number of the first elastic assemblies may be more, which is not limited thereto.

As an optional embodiment, a top portion of the second connecting member 13 is provided with a first protruding portion 131, and the first protruding portion 131 protrudes outward around a circumferential direction of the second connecting member 13. One end of the first elastic member 14 abuts against the first protruding portion 131, and the other end of the first elastic member 14 is connected to the wheel frame plate 3. In some embodiments, one end of the first elastic member 14 may alternatively abuts against the first protruding portion 131, and the other end of the first elastic member 14 is connected to the mounting base 5 or the electromagnet 1. Those skilled in the art can configure a connection mode of the first elastic member 14 and the second connecting member 13 according to the actual situation, which is not limited herein.

Further, referring to FIG. 3 to FIG. 6, in an optional embodiment, the first elastic member 14 may be a single elastic member as described above, or may include a plurality of elastic members. The wheel frame plate 3 is provided with a first through hole 31 and the first groove 32. The second connecting member 13 extends through the first through hole 31, and the first groove 32 surrounds the first through hole 31. The first elastic member 14 extends through the first through hole 31 along the first direction Z. The first elastic member 14 may include a first segment 141 and a second segment 142. One end of the first segment 141 is sleeved on the top portion of the second connecting member 13, and the other end of the first segment 141 is in contact with a bottom wall of the first groove 32. One end of the second segment 142 is in contact with a bottom surface of the wheel frame plate 3, and the other end of the second segment 142 is connected to the mounting base 5 or the electromagnet 1. It should be noted that in the embodiment shown in FIG. 4, the first segment 141 is a part of the first elastic member 14 located above the wheel frame plate 3, one end of the first segment 141 is wound around the top portion of the second connecting member 13, and the other end of the first segment 141 is in contact with the bottom wall of the first groove 32. The second segment 142 is a part of the first elastic member 14 located below the wheel frame plate 3, one end of the second segment 142 is in contact with the bottom surface of the wheel frame plate 3, and the other end of the second segment 142 is connected to the electromagnet 1.

The braking device 8 has a power-off state and a power-on state. When the braking device 8 is in the power-off state, the electromagnet 1 is magnetized, e.g., the electro permanent magnet recovers the magnetization, and is attracted to the braking plate 9. When the electromagnet 1 moves close to the braking plate 9 or swings around the radial direction of the first connecting member 11, the electromagnet 1 drives the mounting base 5 and the second connecting member 13 to move close to the braking plate 9 or swing around the radial direction of the first connecting member 11, so that the second connecting member 13 move downward along the first through hole 31, so that the first segment 141 located between the top portion of the second connecting member 13 and the bottom surface of the first groove 32 is compressed and stores potential energy, and the second segment 142 located between the wheel frame plate 3 and the electromagnet 1 is stretched and stores potential energy, so that the electromagnet 1 gradually comes into contact with the braking plate 9 to achieve braking.

When the braking device 8 is in the power-on state, the electromagnet 1 demagnetizes to be separated from the braking plate 9. As the attraction force between the electromagnet 1 and the braking plate 9 is released, the compressed first segment 141 releases potential energy to provide upward elastic force to drive the top portion of the second connecting member 13 to move upward, and the stretched second segment 142 also releases potential energy to provide restoring force, so as to pull the electromagnet 1 upward under the action of the first elastic member 14 until the first elastic member 14 is reset to separate the electromagnet 1 from the braking plate 9.

It should be noted that when the electromagnet 1 only moves up and down in the direction perpendicular to the braking plate 9, the second connecting member 13 also only moves up and down along the first through hole 31. In that case, the wheel frame plate 3 will not move due to the up-and-down movement of the electromagnet 1 and the second connecting member 13. Meanwhile, when the electromagnet 1 swings around the radial direction of the first connecting member 11, the second connecting member 13 abuts against the side wall of the first through hole 31 and swing around the radial direction of the first connecting member 11, thereby causing the wheel frame plate 3 to swing in the radial direction of the first connecting member 11. In this way, when the roller 2 moves on the bed frame 6, the roller 2 is prevented from being suspended due to an inclination of the bed frame 6.

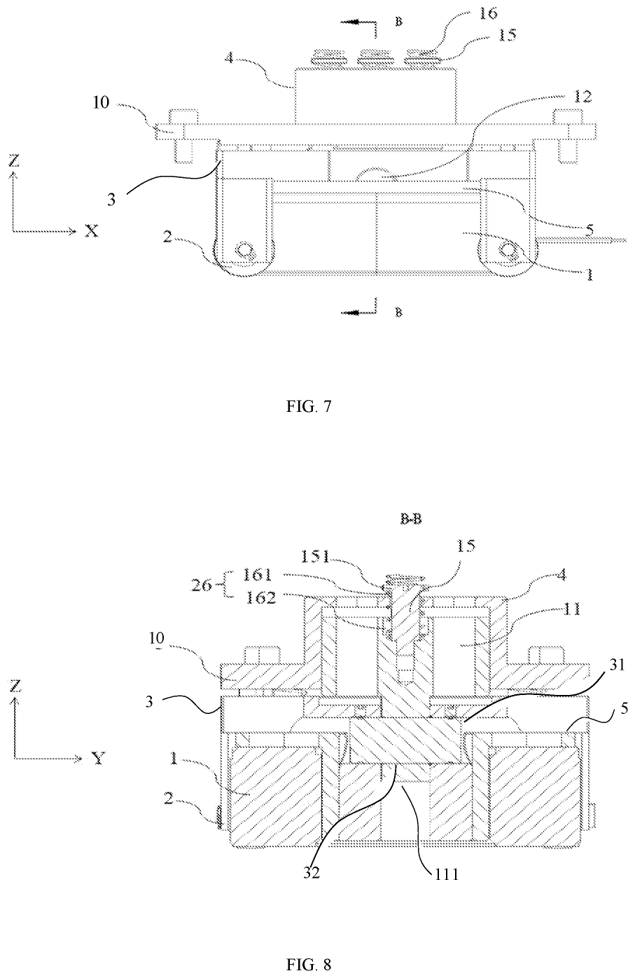

Referring to FIGS. 7 to 8, the braking device 8 further includes the housing 4 and a second elastic assembly. The second elastic assembly includes a third connecting member 15 and a second elastic member 16. The first connecting member 11 extends through the wheel frame plate 3, and the housing 4 is fixed to the connecting plate 10 and covers a portion of the first connecting member 11 located above the wheel frame plate 3. The third connecting member 15 extends through the housing 4, and is in contact with a top portion of the first connecting member 11. The second elastic member 16 is sleeved on the third connecting member 15, and the second elastic member 16 is configured to drive the first connecting member 11, the wheel frame plate assembly and the electromagnet 1 to move closes to or away from the braking plate 9 through the third connecting member 15. With such configuration, when a distance between the electromagnet 1 and the braking plate 9 is relatively large, for example, when the first segment 141 of the first elastic member 16 cannot bring the electromagnet 1 into contact with the braking plate 9 even if the first segment 141 of the first elastic member 16 is completely compressed, the second pressure spring 16 can cooperate with the housing 4 to reduce the distance between the electromagnet 1 and the braking plate 9, so that the electromagnet 1 can swing around the first connecting member 11 in the radial direction to be in contact with the braking plate 9, thereby increasing the contact area between the electromagnet 1 and the braking plate 9 to increase the braking force applied on the braking plate 9.

As an optional embodiment, a top portion of the third connecting member 15 has a second protruding portion 151, and the second protruding portion 151 protrudes outward around a circumferential direction of the third connecting member 15. One end of the second elastic member 16 abuts against the second protruding portion 151, and the other end of the second elastic member 16 is connected to the first connecting member 11. In some embodiments, one end of the second elastic member 16 abuts against the second protruding portion 151, and the other end of the second elastic member 16 is connected to the wheel frame plate 3. Those skilled in the art can configure a connection mode between the second elastic member 16 and the third connecting member 15 according to the actual situation, which is not limited herein. In an embodiment, the braking device 8 includes at least two second elastic assemblies, and at least two second elastic assemblies are evenly distributed on the housing 4 relative to the first connecting member 11. With such configuration, the plurality of second elastic assemblies can apply force to the electromagnet 1 evenly, so that the electromagnet 1 can be pulled up or down smoothly. In the embodiment shown in FIG. 7, the number of the second elastic assemblies is three. In some embodiments, the number of the second elastic assemblies can also be more, and the present disclosure is not limited herein.

Further, similar to the first elastic member 14, the second elastic member 16 may be a single elastic member as described above, or may include a plurality of elastic members. In an embodiment, the second elastic member 16 includes a third segment 161 and a fourth segment 162. One end of the third segment 161 is sleeved on the top portion of the third connecting member 15, and the other end of the third segment 161 is in contact with an outer wall of the housing 4. One end of the fourth segment 162 is in contact with an inner wall of the housing 4, and the other end of the fourth segment 162 is connected to the first connecting member 11. In the embodiment shown in FIG. 8, the third segment 161 is a part of the second elastic member 16 located above the housing 4, one end of the third segment 161 is wound around the top portion of the third connecting member 15, and the other end of the third segment 161 is in contact with the outer wall of the housing 4. The fourth segment 162 is a part of the second elastic member 16 located in the housing 4, one end of the fourth segment 162 is in contact with the inner wall of the housing 4, and the other end of the fourth segment 162 is connected to the first connecting member 11.

When the braking device 8 is in the power-off state, the electromagnet 1 is magnetized, e.g., the electro permanent magnet recovers the magnetization, and the electromagnet 1 is attracted to the braking plate 9. When the electromagnet 1 has a downward movement close to the braking plate 9, the electromagnet 1 first drives the mounting base 5 and the second connecting member 13 to move close to the braking plate 9. In the case the electromagnet 1 cannot come into contact with the braking plate 9 even when the second connecting member 13 moves downward along the first through hole 31 until the first segment 141 is fully compressed, for example, when the distance between the electromagnet 1 and the braking plate 9 is large, the electromagnet 1 will continue to move downwards under the attraction force. At this time, the second connecting member 13 and the wheel frame plate 3 are driven by the electromagnet 1 to move downward. Then the first connecting member 11 and the mounting base 5 move downward. When the first connecting member 11 moves downward, the third segment 161 is compressed and stores potential energy, and the fourth segment 162 is stretched and stores potential energy, so that the entire braking device 8 except the connecting plate 10 and the housing 4 moves close to the braking plate 9 until the electromagnet 1 is in contact with the braking plate 9, thus increasing the moving distance of the electromagnet 1.

When the braking device 8 is in the power-on state, the electromagnet 1 is demagnetized to be separated from the braking plate 9. Since the attraction force between the electromagnet 1 and the braking plate 9 is released, the compressed third segment 161 and the compressed first segment 141 releases potential energy to provide an upward elastic force to drive the third connecting member 15 and the second connecting member 13 to move upward respectively. The stretched fourth segment 162 and the second segment 142 release potential energy to provide restoring force, so as to pull the electromagnet 1 upward under the action of the first elastic member 14 and the second elastic member 16 until the first elastic member 14 and the second elastic member 16 are reset to separate the electromagnet 1 from the braking plate 9.

When the electromagnet 1 has a swing motion around the radial direction of the first connecting member 11 close to the braking plate 9, such as when the electromagnet 1 swings around the rotating axis 12, since the wheel frame plate assembly and the first connecting member 11 swing along with the electromagnet 1, the third connecting member 15 connected to the first connecting member 11 also swing along with the electromagnet 1, and the third connecting member abuts against the housing 4 in the radial direction of the housing 4, causing the housing 4 and the connecting plate 10 to swing accordingly, thereby driving the bed plate 72 to swing, so that the bed plate 72 can be parallel to the braking plate 9. It should be noted that an angle of swing is small in practical applications. The angle of the swing is similar to the preset angle described above.

It should be noted that the electromagnet 1 can simultaneously have the downward movement close to the braking plate 9 and the swing motion around the radial direction of the first connecting member 11 close to the braking plate 9. The above description is for the convenience of explanation, and the two movements are explained separately.

Referring to FIG. 9, the wheel frame plate assembly further includes a wheel frame 21, a connecting shaft 22, a snap ring 23, and a washer 24. The wheel frame 21 extends along the first direction Z, one end of the wheel frame 21 is connected to the wheel frame plate 3, and the other end of the wheel frame 21 is provided with a recessed groove extending along the first direction Z. The connecting shaft 22 extends through the recessed groove. The wheel 2 and the washer 24 are sequentially sleeved on the connecting shaft 22, and the snap spring 23 is arranged on a side wall of the wheel frame 21 and is engaged with the connecting shaft 22. With such configuration, the washer 24 and the wheel 2 are sequentially sleeved on the connecting shaft 22, the washer 24 can prevent the wheel 2 from jumping on the connecting shaft 22, and the snap spring 23 is arranged on the side wall of the wheel frame 21 for fixing the connecting shaft 22 to prevent the connecting shaft 22 from falling off from the wheel frame 21. In some embodiments, the wheel 2 may also include other components with the same function, which is not limited herein.

Referring to FIGS. 1 to 9, a working principle of the braking device 8 of the present disclosure will be further described below. When the braking device 8 is in the power-on state, the electromagnet 1 is demagnetized to be separated from the braking plate 9. Since the attraction force between the electromagnet 1 and the braking plate 9 is released, the first elastic member 14 and the second elastic member 16 are converted from the potential energy storing state to the potential energy releasing state. Specifically, the compressed third segment 161 and the compressed first segment 141 release potential energy to provide upward elastic force to respectively drive the third connecting member 15 and the second connecting member 13 to move upward, and the stretched fourth segment 162 and the second segment 142 releases potential energy to provide an upward restoring force, so that the electromagnet 1 is pulled upward under the force of the first elastic member 14 and the second elastic member 16 until the first elastic member 14 and the second elastic member 16 are reset to separate the electromagnet 1 from the braking plate 9. It should be noted that when the braking device 8 is in a power-off state, only the first elastic member may be in the energy storing state, while the second elastic member is in the normal state. In this case, the compressed first segment 141 releases the potential energy is to provide an upward elastic force, so as to drive the second connecting member 13 to move upward, and the stretched second segment 142 releases the potential energy to provide an upward restoring force, so that the electromagnet 1 is pulled upward under the action of the first elastic member 14. Until the first elastic member 14 is reset, the electromagnet 1 is separated from the braking plate 9.

When the braking device 8 is in the power-off state, the electromagnet 1 is magnetized. When there is a small gap between the electromagnet 1 and the braking plate 9, the electromagnet 1 moves downward close to the braking plate 9, and the electromagnet 1 drives the mounting base 5 and the second connecting member 13 to move close to the braking plate 9, causing the second connecting member 13 moves downward along the first through hole 31, so that the first elastic member stores potential energy. Specifically, the first segment 141 is compressed and stores potential energy, and the second segment 141 is stretched and stores potential energy, so that the electromagnet 1 can be in contact with the braking plate 9. When the electromagnet 1 cannot be completely attached to the braking plate 9, for example, the mounting surface is not parallel to the braking plate 9, since part of the electromagnet 1 is in contact with the braking plate 9 and another part of the electromagnet 1 is suspended, the electromagnet 1 will swing around the radial direction of the first connecting member 11, such as the swing of the rotating shaft 12, under the action of the attraction force, thereby increasing the contact area between the electromagnet 1 and the braking plate 9, and even achieving complete attachment between the electromagnet 1 and the braking plate 9, thereby increasing a braking force provided by the braking device 8 on the braking plate 9.

In the case that before the electromagnet 1 is magnetized and the electromagnet is partially in contact with the braking plate 9, the contact area between the electromagnet 1 and the braking plate 9s 9 can be increased by swinging the electromagnet 1 around the radial direction of the first connecting member 11, thereby increasing a braking force provided by the braking device 8 on the braking plate 9.

In the case that before the electromagnet 1 is magnetized and there is a large gap between the electromagnet 1 and the braking plate 9, for example, the electromagnet 1 cannot come into contact with the braking plate 9 even when the second connecting member 13 moves downward along the first through hole 31 until the first segment 141 is fully compressed, the electromagnet 1 will continue to move downward under the action of the attraction force. At this time, the second connecting member 13 and the wheel frame plate 3 are driven by the electromagnet 1 to move downward. Then the first connecting member 11 and the mounting base 5 move downward. When the first connecting member 11 moves downward, the third segment 161 is compressed and stores potential energy, and the fourth segment 162 is stretched and stores potential energy, so that the entire braking device 8 except the connecting plate 10 and the housing 4 moves close to the braking plate 9 until the electromagnet 1 is in contact with the braking plate 9. When the electromagnet 1 comes into contact with the braking plate 9, the electromagnet 1 swings around the radial direction of the first connecting member 11 to increase the contact area between the electromagnet land the braking plate 9, thereby increasing a braking force provided by the braking device 8 on the braking plate 9.

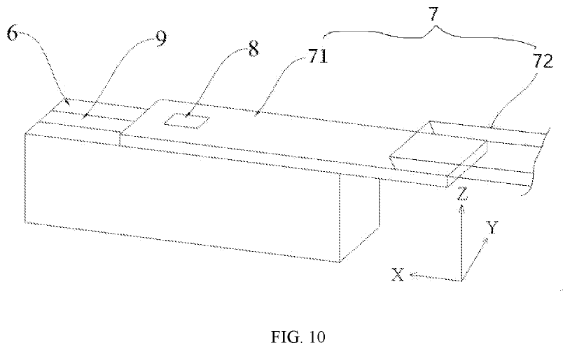

Referring to FIG. 10, the present disclosure further provides an examination bed, which includes a bed frame 6, a moving member 71, a bed plate 72 configured to carry a patient and connected to the moving member 71, and the braking device 8 as described above. The braking plate 9 is fixed to the bed frame 6. The braking device 8 is provided between the moving member 7 and the braking plate 9 and is opposite to the braking plate 9 for braking the braking plate 9. The moving member 7 is configured to be driven by a power to move back and forth on the bed frame 6, thereby driving the braking device 8 and the bed plate 72 to move back and forth on the bed frame 6. In an embodiment, the braking device 8 is detachably connected to the moving member 7. With such configuration, the patient lying on the bed plate 72 is transported to a detection position through the rolling of the wheel 2 on the braking plate 9, and the braking force is provided through the attraction action between the electromagnet 1 and the braking plate 9, so that the bed plate 72 is braked after the patient is transported to the examination position to prevent the bed plate from moving again to bring harm to the patient.

In summary, in the braking device 8 and the examination bed provided by the embodiments of the present disclosure, the braking device 8 includes the wheel frame plate assembly and the electromagnet assembly, and the wheel frame plate assembly includes the wheel frame plate 3 and the wheel 2. One side of the wheel frame plate 3 along the first direction Z is connected to the wheel 2, and the other side of the wheel frame plate 3 is configured to be connected to the examination bed. The wheel 2 is rotatable and adapted to be disposed above a braking plate 9. The first direction Z is perpendicular to the plane where the wheel frame plate 3 is located. The electromagnet assembly includes the electromagnet 1 and the first connecting member 11. The electromagnet 1 is connected to the wheel frame plate 3. The electromagnet 1 and the wheel 2 are located on the same side of the wheel frame plate 3 along the first direction Z. The electromagnet 1 is attracted to or separated from the braking plate 9 by moving along an axial direction of the first connecting member 11, so as to brake or release the braking force.

The electromagnet 1 can rotate around the radial direction of the first connecting member, such as the rotation axis 12. With such configuration, when the mounting surface of the braking device 8 is not parallel to the braking plate 9, the first connecting member 11 can rotate around the rotating shaft 12 to drive the electromagnet 1 to rotate around the rotating shaft 12, thereby increasing the contact area between the electromagnet 1 and the braking plate 9, and further increasing the braking force exerted on the braking plate 9.

The foregoing descriptions are merely specific embodiments of the present disclosure, but are not intended to limit the protection scope of the present disclosure. Any variation or replacement readily figured out by a person skilled in the art within the technical scope disclosed in the present disclosure shall all fall within the protection scope of the present disclosure.

What is claimed is:

1. A braking device, comprising:
a wheel frame plate assembly, comprising a wheel frame plate and a wheel, one side of the wheel frame plate along a first direction being connected to the wheel, and the wheel being rotatable and adapted to be disposed above a braking plate;
a first connecting member connected to the wheel frame plate assembly; and
an electromagnet assembly comprising an electromagnet, the electromagnet being connected to the wheel frame plate and being rotatable around a radial direction of the first connecting member, and the electromagnet and the wheel being located on the same side of the wheel frame plate along the first direction;
wherein the electromagnet is adapted to be attracted to or separated from the braking plate by moving along an axial direction of the first connecting member, so as to brake or release braking force on the braking plate.

2. The braking device according to claim 1, further comprising a rotating shaft, wherein the rotating shaft extends through the first connecting member in a direction perpendicular to the first connecting member, and both ends of the rotating shaft are respectively connected to the wheel frame plate.

3. The braking device according to claim 1, further comprising a first elastic assembly, wherein the first elastic assembly comprises a second connecting member and a first elastic member, the second connecting member movably extends through the wheel frame plate, a bottom portion of the second connecting member is connected to the electromagnet, and the first elastic member is sleeved on the second connecting member.

4. The braking device according to claim 3, wherein a top portion of the second connecting member is provided with a first protruding portion, and the first protruding portion protrudes outward around a circumferential direction of the second connecting member, one end of the first elastic member abuts against the first protruding portion, and the other end of the first elastic member is connected to the wheel frame plate.

5. The braking device according to claim 3, wherein the wheel frame plate is provided with a first groove, the first elastic member is sleeved on a rod portion of the second connecting member, one end of the first elastic member abuts against a head portion of the second connecting member, and the other end of the first elastic member is located in the first groove and abuts against a bottom wall of the first groove; or, a portion of the first elastic member is wound around an upper portion of the second connecting member, and another portion of the first elastic member and the second connecting member extend into the first groove and are connected to the wheel frame plate.

6. The braking device according to claim 5, wherein the wheel frame plate is further provided with a first through hole, the second connecting member extends through the first through hole, the first groove surrounds the first through hole, the first elastic member extends through the first through hole along the first direction, the first elastic member comprises a first segment and a second segment, one end of the first segment is sleeved on the top portion of the second connecting member, the other end of the first segment is in contact with a bottom wall of the first groove, one end of the second segment is in contact with a bottom wall of the wheel frame plate, the other end of the second segment is connected to the electromagnet.

7. The braking device according to claim 3, wherein the braking device has a power-off state and a power-on state;
when the braking device is in the power-off state, the electromagnet is magnetized and is attracted to the braking plate, and the first elastic member is deformed and stores potential energy; and
when the braking device is in the power-on state, the electromagnet demagnetizes, and separate from the braking plate, and the first elastic member resets and releases potential energy, thereby driving the electromagnet to move away from the braking plate.

8. The braking device according to claim 1, further comprising a housing and a second elastic assembly;
wherein the second elastic assembly comprises a third connecting member and a second elastic member, the first connecting member extends through the wheel frame plate, the housing covers a portion of the first connecting member located above the wheel frame plate;
the third connecting member extends through the housing, and is connected to a top portion of the first connecting member;
the second elastic member is sleeved on the third connecting member.

9. The braking device according to claim 8, wherein a top portion of the third connecting member has a second protruding portion, the second protruding portion protrudes outward around a circumferential direction of the third connecting member, one end of the second elastic member is connected to the housing, and the other end of the second elastic member is connected to the first connecting member.

10. The braking device according to claim 9, wherein the second elastic member comprises a third segment and a fourth segment, one end of the third segment is connected to the second protruding portion, the other end of the third segment is connected to the housing, one end of the fourth segment is connected to the housing, and the other end of the fourth segment is connected to the first connecting member.

11. The braking device according to claim 10, wherein the braking device has a power-off state and a power-on state;
when the braking device is in the power-off state, the electromagnet is magnetized, and the second elastic member is deformed and stores potential energy; and
when the braking device is in the power-on state, the electromagnet is demagnetized, and separate from the braking plate, the second elastic member resets and releases potential energy, thereby driving the electromagnet to move away from the braking plate.

12. The braking device according to claim 1, wherein the electromagnet assembly further comprises a mounting base, the mounting base is arranged between the wheel frame plate and the electromagnet and is fixed to the electromagnet, and the second connecting member is connected to the mounting base.

13. The braking device according to claim 1, wherein the wheel frame plate assembly further comprises a wheel frame and a connecting shaft, the wheel frame extends along the first direction, one end of the wheel frame is connected to the wheel frame plate, another end of the wheel frame is provided with a recessed groove extending along the first direction, the connecting shaft extends through the recessed groove, the wheel is connected to the wheel frame.

14. The braking device according to claim 1, wherein the wheel frame plate assembly further comprises a snap ring, and the snap ring is arranged on a side wall of the wheel frame and is engaged with the connecting shaft.

15. A braking device, comprising:
a wheel frame plate assembly, comprising a wheel frame plate and a wheel, one side of the wheel frame plate along a first direction being connected to the wheel, and the wheel being rotatable and adapted to be disposed above a braking plate;
a first elastic assembly; and
an electromagnet assembly connected to the wheel frame plate through the first elastic assembly, and the electromagnet assembly and the wheel being located on the same side of the wheel frame plate;
wherein the first elastic assembly is capable of deforming or resetting based on movement of the electromagnet assembly in a direction close to or away from the braking plate, so as to enable the braking device to complete braking or release braking.

16. The braking device according to claim 15, wherein the first elastic assembly comprises a second connecting member and a first elastic member, the second connecting member movably extends through the wheel frame plate, a bottom portion of the second connecting member is connected to the electromagnet assembly, and the first elastic member is sleeved on the second connecting member.

17. The braking device according to claim 16, wherein a top portion of the second connecting member is provided with a first protruding portion, and the first protruding portion protrudes outward around a circumferential direction of the second connecting member, one end of the first elastic member abuts against the first protruding portion, and the other end of the first elastic member is connected to the wheel frame plate.

18. The braking device according to claim 17, further comprising a housing and a second elastic assembly;
wherein the second elastic assembly comprises a third connecting member and a second elastic member, the first connecting member extends through the wheel frame plate, the housing covers a portion of the first connecting member located above the wheel frame plate;
the third connecting member extends through the housing, and is connected to a top portion of the first connecting member;
the second elastic member is sleeved on the third connecting member.

19. The braking device according to claim 18, further comprising a first connecting member and a rotating shaft;
wherein the rotating shaft extends through the first connecting member in a direction perpendicular to the first connecting member, and both ends of the rotating shaft are connected to the wheel frame plate respectively to enable the first connecting member and the wheel frame plate to be rotatable with each other.

20. An examination bed, comprising a bed frame, a bed plate assembly, and the braking device according to claim 1, wherein the braking plate is fixed inside the bed frame, the braking device is located between the bed plate assembly and the braking plate, and the braking device is fixed to the bed plate assembly through a fastener.
